(19) United States
(12) Patent Application Publication
(10) Pub. No.: US 2009/0137517 A1
(43) Pub. Date: May 28, 2009

(54) SENSITIZING A CELL TO CANCER TREATMENT BY MODULATING THE ACTIVITY OF A NUCLEIC ACID ENCODING RPS27L PROTEIN

(75) Inventor: Qiang Yu, Genome (SG)

Correspondence Address: CHOATE, HALL & STEWART LLP, TWO INTERNATIONAL PLACE, BOSTON, MA, 02110, US

(73) Assignee: Agency for Science, Technology and Research, Centros (SG)

(21) Appl. No.: 12/281,423

(22) PCT Filed: Mar. 1, 2007
(86) PCT No.: PCT/SG07/00058

§ 371 Date: Nov. 18, 2008

Related U.S. Application Data
(60) Provisional application No. 60/778,519, filed on Mar. 2, 2006.

(51) Int. Cl. A61K 31/7105; C12N 5/06; C12N 15/00

(52) U.S. Cl. 514/44; 435/375; 435/350; 435/354; 435/351; 435/320.1

(57) ABSTRACT

The present invention refers to a method of sensitizing a cell to cancer treatment comprising modulating the activity of a nucleic acid which encodes the RPS27L protein by use of an oligonucleotide which is a RNAi agent or an antisense nucleotide molecule. The oligonucleotides of the present invention can be used in combination with at least one cytostatic drug for, e.g., chemotherapy. The present invention further refers to an expression vector comprising an oligonucleotide used in the method of the present invention as well as to a pharmaceutical comprising the oligonucleotides together with at least one chemotherapeutic agent used in the method of the present invention.

### CROSS-REFERENCE TO RELATED APPLICATIONS

The present application claims the benefit of U.S. provisional application 60/778,519, filed Mar. 2, 2006, the entire contents of which is incorporated by reference herein for all purposes.

### FIELD OF THE INVENTION

The present invention refers to a method of sensitizing a cell to cancer treatment comprising administering to a cell a compound (capable of) modulating the activity of a nucleic acid which encodes the RPS27L protein. Compounds capable of such modulation include oligonucleotides which can for example, be RNAi agents or antisense nucleotide molecules. Such oligonucleotides disclosed in the present invention can be used in combination with at least one cytostatic drug for, e.g., chemotherapy. The present invention further refers in exemplary embodiments to an expression vector comprising an oligonucleotide used in the present invention as well as to a pharmaceutical composition comprising such oligonucleotides together with at least one chemotherapeutic agent of the present invention.

### BACKGROUND OF THE INVENTION

Cancer is pathological disorder in which a group of cells (usually derived from a single cell) have lost their normal growth control mechanisms and thus show unregulated growth. Cancerous (malignant) cells can develop from any tissue within any organ. As cancerous cells grow and multiply, they form a mass of cancerous tissue—called a tumor—that invades and destroys normal adjacent tissues. The term “tumor” refers to an abnormal growth or mass; tumors can be cancerous or noncancerous. Cancerous cells from the primary (initial) site can spread (metastasize) throughout the body. Cancer is a major cause of death worldwide, being the second-leading cause of death in developed countries and even the number one cause of death in e.g. Australia, Japan, Korea, Singapore and the male population of the UK and Spain. The number of people who develop cancer each year is continuously increasing.

Presently available drugs used for treating cancer aim at causing the specific death of those cells forming the tumor. According to the present tenet of oncology tumor (malignant) cells treated with anticancer drugs in chemotherapy die from apoptosis. Apoptosis is a distinct mode of cell death that is responsible for deletion of cells in normal tissues; it also occurs in specific pathologic contexts. Morphologically, it involves rapid condensation and budding of the cell, with the formation of membrane-enclosed apoptotic bodies containing well-preserved organelles, which are phagocytosed and digested by nearby resident cells. There is no associated inflammation. A characteristic biochemical feature of the process is double-strand cleavage of nuclear DNA at the linker regions between nucleosomes leading to the production of oligonucleosomal fragments. In many, although not all of the circumstances in which apoptosis occurs, it is suppressed by inhibitors of messenger RNA and protein synthesis. Apoptosis occurs spontaneously in malignant tumors, often markedly retarding their growth, and it is increased in tumors responding to irradiation, cytotoxic chemotherapy, heating and hormone ablation. However, much of the current interest in the process stems from the discovery that it can be regulated by certain proto-oncogenes and the p53 tumor suppressor gene. The p53 tumor suppressor is required for efficient execution of the death program.

To initiate the p53 dependent death program of the cell, patients are subjected, for example, to chemotherapy with different kinds of cytotoxic drugs. Although an ideal chemotherapy drug would destroy cancer cells without harming normal cells, few such drugs exist. Instead, in present chemotherapy, drugs are designed to inflict greater damage on cancer (malignant) cells than on normal non-malignant cells. Nonetheless, all chemotherapy drugs affect normal cells and cause side effects. Thus, there is a need to develop further methods for the treatment of cancer which cause lesser side effects.

Accordingly it is an object of the present invention to provide a method that is capable of killing cancer (malignant) cells without causing too much or no side effects.

### SUMMARY OF THE INVENTION

In one aspect the present invention relates to a method of sensitizing a cell to cancer treatment comprising administering to a cell a compound modulating the activity of a nucleic acid which encodes the RPS27L protein or the RPS27L protein itself.

In one aspect modulating the activity of the nucleic acid which encodes the RPS27L protein comprises administering to a subject a compound such as a nucleic acid molecule. Examples of suitable nucleic acid molecules that are able to modulate the activity of a nucleic acid molecule comprise an oligonucleotide such as a RNAi agent or an antisense nucleotide molecule.

In still another aspect, this method further comprises administering at least one chemotherapeutic agent.

The present invention also relates to an expression vector comprising at least one oligonucleotide used in the method of the present invention.

In another aspect the present invention relates to a pharmaceutical preparation comprising at least one chemotherapeutic agent used in the method of the present invention together with at least one compound, for example at least one RNAi agent or/and at least one antisense nucleotide molecule used in the method of the present invention.

### BRIEF DESCRIPTION OF THE DRAWINGS

The invention will be better understood with reference to the detailed description when considered in conjunction with non-limiting examples and the accompanying drawings, in which:

FIG. 1 shows that RPS27L is a direct transcriptional target of p53 (for further details see also Example 1). FIG. 1A shows a microarray analysis demonstrating that the expression of RPS27L was induced by the DNA damaging agents adriamycin (ADR) and 5-fluorouracil (5-FU) in p53 wild-type HCT116 cells. In general, FIG. 1A depicts a cluster diagram of microarray data showing genes upregulated by the genotoxic agents 5-fluorouracil (5FU) and adriamycin (ADR) in a p53-dependent manner. FIG. 1B shows treatment of p53+/+ and p53−/− HCT116 cells with ADR (1 μM) or 5-FU (375 μM) for the indicated times. RPS27L levels were determined by RT-PCR. GAPDH was used as loading control. FIG. 1B shows that RPS27L mRNA was induced following ADR or 5-FU treatment only in p53 wild-type HCT116 cells but not in cells which are p53 negative. These results demonstrate that there is a connection between the expression of RPS27L and the activation and expression of p53. FIG. 1C demonstrates that p53 binds to the first intron of the RPS27L gene. Genome-wide p53 binding targets in HCT116 cells have previously been performed using ChIP-PET technology (Wei, C. L., Wu, Q., et al., (2006) “A global map of p53 transcription-factor binding sites in the human genome” Cell, vol. 124, p. 207-219). Illustrated are the nine PETs binding to the first intron of the RPS27L gene in HCT116 cells treated with 5-FU. The overlapped region contains a consensus p53 binding motif. These results demonstrate that RPS27L expression appears to be up-regulated by p53 through direct DNA binding. FIG. 1D shows that p53 activates the RPS27L gene promoter containing the p53 binding site. Upper panel, schematic structure of the RPS27L gene promoter. Two luciferase reporter constructs containing the putative p53 binding sites within the 1.1 kb RPS27L promoter region (fragment A) and the region containing the ChIP-validated p53 binding site (fragment B) were constructed. p53 RE, p53 response elements; Lower panel, the above constructs were co-transfected with wild-type p53 and the DNA-binding mutant p53 (175H) and luciferase activity was measured. A reporter construct containing the p21 promoter was used as a positive control. These results confirm that the p53 binding located in the first intron, as determined by ChIP analysis, is functional and confers the p53 responsiveness.

FIG. 2 shows Western Blot analysis demonstrating that RPS27L protein is differentially expressed in response to distinct stress signals (for further details see Example 2). FIG. 2A shows the results of the treatment of p53+/+ and p53−/− HCT116 cells with ADR (1 μM), 5-FU (375 μM) and nutlin-3 (10 μM) for the indicated times. p53, RPS27L and p21 protein levels were determined by Western blot analysis. Tubulin was examined as a loading control. It can be taken from FIG. 2A that the protein levels of RPS27L increased when the cells were treated with ADR and nutlin-3. FIG. 2B shows the result of treatment of U2OS, Saos-2 and SH-SY5Y cells with etoposide phosphate (VP16®) (10 μM), ADR (1 μM) or 5-FU (375 μM) for the indicated times. p53, p21 and RPS27L protein levels were determined by Western blot analysis. Tubulin was examined as a loading control. Again, RPS27L protein levels increased upon treatment of the cells with ADR and etoposide phosphate (VP16®).

FIG. 3 demonstrates that RPS27L expression modulates p53-dependent apoptosis (for further details see Example 3). FIG. 3A shows cell cycle and apoptosis analysis in HCT116 cells (p53+/+ and p53−/−), as measured by flow cytometry. Cells were treated with ADR (1 μM) or 5-FU (375 μM) for 48 h. FIG. 3A demonstrates that the DNA damaging agent ADR induces p53-dependent cell cycle arrest (increased number of hyperploid cells (4N)), whereas 5-FU treatment triggers p53-dependent apoptosis. FIG. 3B shows the results of transfection of HCT116 cells with RPS27L siRNA or a control siRNA, followed by etoposide phosphate (VP16®) (10 μM) treatment for indicated times. RPS27L, RPS27 and GAPDH mRNA levels were determined by RT-PCR. FIG. 3B shows that the targeted sequence for siRNA was efficient and specific as it nearly completely ablated RPS27L expression and prevented its induction after DNA damage, while having no effect on closely-related RPS27. FIG. 3C shows treatment of HCT116 cells, which stably express the RPS27L shRNA or a control shRNA, with ADR (1 μM) for 48 hours. Cell death (apoptosis) was measured by cells with sub-G1 DNA content. The bar graph shows the averaged results of three independent experiments with standard deviation (s.d). indicated. In p53 wild-type HCT116 cells expressing the control shRNA, ADR treatment for 48 h resulted primarily in a growth arrest response, while RPS27L-depleted cells also receiving ADR underwent marked cell death. FIG. 3D shows the result of transfection of HCT116 cells with RPS27L siRNA, followed by etoposide phosphate (VP16®) (10 μM) or ADR (1 μM) treatment for 24 hours. Cell death was measured by cells with sub-G1 DNA content. Each bar represents the mean±s.d. of three independent experiments. FIG. 3D shows that knockdown of RPS27L through transient siRNA transfection also induced a marked increase in cell death upon ADR or etoposide phosphate (VP16®) treatment in HCT116 cells but not in p53 null counterparts. FIG. 3E shows cells which have been treated as the cells depicted in FIG. 1C, followed by JC-1 staining and flow cytometry analysis. Mitochondrial impairment was represented as the percentage of cells with lower membrane potential (ΔΨm). The bar graph shows the results of three independent experiments. FIG. 3E illustrates that ADR treatment of RPS27L shRNA cells resulted in a marked decrease in ΔΨm compared to the control cells (33.5% versus 13.5%), indicating an apoptotic cell death involving mitochondrial dysfunction.

FIG. 4 demonstrates that RPS27L is a nuclear protein that forms DNA damage foci upon DNA damage (for further details see Example 4). FIG. 4A shows HCT116 cells transfected with a Myc-tagged RPS27L expression vector. The transfected cells were fixed and stained with anti-Myc antibody and FITC-conjugated anti-mouse Ig (green), followed by confocal microscopy examination. Nuclei were stained with DRAQ5 (blue). Rodamine-Phalloiadin was used for counter-staining of the actin cytoskeleton (red). FIG. 4B shows HCT116 control and RPS27L-depleted cells treated with VP16 (20 μM) for 16 hours. Co-localization of RPS27L with γ-H2AX or TopBP1 was detected by immunofluorescence staining with anti-RPS27L (green) and anti-γ-H2AX or anti-TopBP1 (red). Nuclei were stained by DRAQ5 (blue).

FIG. 5 shows that RPS27L deficiency results in defective cell cycle checkpoint and DNA repair, i.e. loss of RPS27L leads to chromosome instability (for further details see Example 5). FIG. 5A shows HCT116 control and RPS27L-depleted cells treated with ADR (1 μM) for 24 hours. Cells were labeled with BrdU for 30 min and stained with FITC-conjugated anti-BrdU and 7-AA-D. Incorporation of BrdU (y axis) and total DNA content (x axis) were analyzed by flow cytometry. The representative histogram indicates the percentages of cells in S-phase. The bar graphs show the results of three independent experiments. FIG. 5B shows HCT116 control and RPS27L-depleted cells treated with etoposide phosphate (VP16®) (20 μM) for 3 hours. Thereafter, etoposide phosphate (VP16®) was removed by replacing with fresh medium and cells were harvested at 0, 3, 6 and 16 hours for anti-γH2AX staining. Stained cells were examined by confocal microscopy. Nuclei were stained with DRAQ5. FIG. 5C shows DNA damage as measured by the comet assay in HCT116 control and RPS27L-depleted cells after 24 h of ADR treatment. The damage distribution, measured as tail moment (product of tail length and fraction of DNA), differed between the two cell lines. Tail moment (in microns) after treatment is given. FIG. 5D show the numbers of micronuclei (percentage) measured by the CBMN assay after a 24 h ADR treatment. Data shown is for ADR treatment (1 μM) of HCT116 control and RPS27L-depleted cells in comparison with the untreated samples. A total of 1000 binucleated cells were scored.

FIG. 6 depicts Western Blot analysis showing that RPS27L depletion impairs p21 accumulation upon DNA damage (for further details see Example 6). FIG. 6A shows Western Blot analysis of expression levels of p53 and its target genes p21, Puma and MDM2 in HCT116 control and RPS27L-depleted cells treated with ADR (1 μM) for 24 and 48 hours. Tubulin was used as a loading control. FIG. 6B shows analysis of U2OS cells transfected with RPS27L siRNA or the control siRNA. The transfected cells were treated with ADR (1 μM) for 24 hours. The expression levels of p53 and its target genes are shown as in FIG. 6A. FIG. 6C shows RT-PCR analysis of RPS27L and p21 mRNA levels in HCT116 control and RPS27L-depleted cells treated with ADR as indicated. FIG. 6D shows results of protein analysis of HCT116 cells transfected with p21 and increasing amounts of RPS27L. p21 and RPS27L protein expression was examined by Western blot analysis. Actin was used a loading control.

FIG. 7 demonstrates that p21 depletion is sufficient to induce a cell cycle checkpoint defect and increased apoptosis in response to DNA damage (for further details see Example 6). FIG. 7A shows Western Blot analysis for HCT116 control and p21-depleted cells treated with ADR for 24 h and for cell lysates prepared for Western blot analysis of p53, p21 and Puma. FIG. 7B shows HCT116 control and p21-depleted cells treated with ADR for 24 h and stained with BrdU and 7-AA-D for DNA synthesis and DNA content, respectively. Stained cells were analyzed by FACS. Histograms indicate the percentages of cells in S-phase and cell population with DNA content >4N. The bar graphs show the results of three independent experiments. FIG. 7C shows cells treated as the cells depicted in FIG. 7A and cell death was assessed as cells with sub-G1 content. FIG. 7D shows a model of RPS27L function in DNA damage response. Induction of RPS27L by p53 protects against DNA damage through p21-dependent and independent mechanisms.

### DEFINITIONS

Certain terms used in the present specification, examples, and appended claims are explained in more detail in the following. Unless defined otherwise, all technical and scientific terms used herein have the same meaning as is commonly understood by one of ordinary skill in the art to which this invention belongs. Likewise, the terminology used herein describes particular embodiments only, and is not intended to limit the scope of the invention. All documents which are cited within the application are incorporated herein by reference.

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

The terms “to administer” or “administration” refer to the delivery of compounds/molecules referred to in the present invention, such as a therapeutically active nucleic oligonucleotides or chemotherapeutic agents, or of a pharmaceutical composition containing the molecules referred to in the present invention to an organism for the purpose of prevention or treatment of cancer or other disorders which can be influenced by modulating the expression of RPS27L.

The term “transfection” means the introduction of a nucleic acid, e.g., an expression vector, into a recipient cell by nucleic acid-mediated gene transfer.

The term “vector” refers to a nucleic acid molecule capable of transporting another nucleic acid to which it has been linked. One type of vector is a genomic integrated vector, or “integrated vector”, which can become integrated into the chromosomal DNA of the host cell. Another type of vector is an episomal vector, i.e., a nucleic acid capable of extra-chromosomal replication in an appropriate host, e.g., a eukaryotic or prokaryotic host cell. Vectors capable of directing the expression of genes to which they are operatively linked are referred to herein as “expression vectors”. In the present specification, “plasmid” and “vector” are used interchangeably unless otherwise clear from the context.

The term “nucleic acid”, “nucleotide”, “nucleotide molecule” or “oligonucleotide” refers to polynucleotides such as deoxyribonucleic acid (DNA), and ribonucleic acid (RNA). The term should also be understood to include, as applicable to the embodiment being described, single-stranded (such as sense or antisense) and double-stranded polynucleotides. Besides ribose or deoxyribose the sugar groups of the nucleotide subunits may be also modified derivatives thereof such as 2′-O-methyl ribose. The nucleotide subunits of an oligonucleotide may be joined by phosphodiester linkages, phosphorothioate linkages, methyl phosphonate linkages or by other rare or non-naturally-occurring linkages that do not prevent hybridization of the oligonucleotide. Furthermore, an oligonucleotide may have uncommon nucleotides or non-nucleotide moieties.

With the phrase “nucleic acid hybridization” is meant the process by which two nucleic acid strands having completely or partially complementary nucleotide sequences come together under predetermined reaction conditions to form a stable, double-stranded hybrid with specific hydrogen bonds. Either nucleic acid strand may be a deoxyribonucleic acid (DNA), a ribonucleic acid (RNA), or an analog of one of these nucleic acids; thus hybridization can involve RNA:RNA hybrids, DNA:DNA hybrids, or RNA:DNA hybrids.

By “complementary” is meant that the nucleotide sequences of similar regions of two single-stranded nucleic acids, or to different regions of the same single-stranded nucleic acid have a nucleotide base composition that allow the single strands to hybridize together in a stable double-stranded hydrogen-bonded region. When a contiguous sequence of nucleotides of one single-stranded region is able to form a series of “canonical” hydrogen-bonded base pairs with an analogous sequence of nucleotides of the other single-stranded region, such that A is paired with U or T and C is paired with G, the nucleotides sequences are “perfectly” complementary.

A “protein coding sequence” or a sequence that “encodes” a particular polypeptide or peptide, is a nucleic acid sequence that is transcribed (in the case of DNA) and is translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to cDNA from eukaryotic mRNA, genomic DNA sequences from eukaryotic DNA, and even synthetic DNA sequences. A transcription termination sequence will usually be located 3′ to the coding sequence.

“Cells” or “host cells” are terms used interchangeably herein. It is understood that such terms refer not only to the particular subject cell but to the progeny or potential progeny of such a cell.

The term “sensitizing” as used in the specification and the claims of the present invention describes that a cell subjected to the method of the present invention is more susceptible to a certain treatment than before. For example, a therapeutic method for the treatment of cancer using chemotherapeutic agents described herein, wherein the chemotherapeutic agent had no effect or only at higher dosages, could be used or used at lower doses after “sensitizing” the cell using the method of the present invention.

With “modulating the activity of a nucleic acid” is meant altering or modulating, for example decreasing or increasing the transcription level and/or translation (expression) level of a coding sequence, e.g., genomic DNA, mRNA etc., into a polypeptide, for example protein, product.

### DETAILED DESCRIPTION OF THE INVENTION

In response to DNA damage, mammalian cells will activate a protection system to enable repair in order to continue the normal life cycle, or they may, as previously already mentioned, activate the apoptotic machinery in the face of excessive and irreparable damage (Zhou, B. B. and Elledge, S I J. (2000) “The DNA damage response: putting checkpoints in perspective” Nature, vol. 408, p. 433-439). The tumor suppressor p53 is believed to play important roles in DNA damage response. As a transcription factor with DNA-binding activity, p53 binds to as many as 300 target genes in the human genome (Wei, C. L., Wu, Q. et al. (2006) “A global map of p53 transcription-factor binding sites in the human genome” Cell, vol. 124, p. 207-219) and actively regulates the expression of its downstream target genes (Kho, P. S., Wang, Z. et al. (2004) “p53-regulated transcriptional program associated with genotoxic stress-induced apoptosis” J Biol Chem, vol. 279, p. 21183-21192). Major consequences of p53 activation following DNA damage are the induction of cell-cycle arrest, senescence or apoptosis (Lane, D. P. and Lain, S. (2002) “Therapeutic exploitation of the p53 pathway” Trends Mol Med, vol. 8, p. 38-42; Vogelstein, B., Lane, D. and Levine, A. J. (2000) “Surfing the p53 network” Nature, vol. 408, p. 307-310).

Current evidence suggests that p53-dependent cell cycle arrest is primarily mediated through transcriptional induction of the cyclin-dependent kinase (CDK) inhibitor p21 (el-Deiry, W. S., Tokino, T., et al., (1993) “WAF1, a potential mediator of p53 tumor suppression” Cell, vol. 75, p. 817-825; Harper, J. W., Adami, G. R., et al., (1993) “The p21 Cdk-interacting protein Cip1 is a potent inhibitor of G1 cyclin-dependent kinases” Cell, vol. 75, p. 805-816). The mechanism for p53-induced apoptosis, however, is less clear. It has been thought that p53 induces apoptosis through transcriptional activation of apoptotic target genes, such as PUMA, BAX, NOXA, BID, PIG3, CD95, DR5 or p53AIP1 (Vogelstein, B., Lane, D. and Levine, A. J. (2000), supra; Vousden, K. H. and Lu, X. (2002) “Live or let die: the cell's response to p53” Nat Rev Cancer, vol. 2, p. 594-604).

The cellular response to p53 activation following DNA damage varies by cell type and stimuli. The response could be the initiation of DNA repair and the damage checkpoint, leading to the cell cycle arrest, or apoptosis as a result of defective DNA repair. For example, activation of p53 by the DNA damaging agent adriamycine (ADR or also referred to as doxorubicine) resulted in p53-dependent cell cycle arrest in HCT116 cells (human colorectal carcinoma cell line containing wild type p53), whereas in the same cells p53 activation by the DNA analog 5-Fluorouracil give rise to apoptosis. The signals governing cell fate following p53 response to different genotoxic stresses are not yet understood.

Furthermore, increasing evidence suggests that p53-dependent transcription often elicits an anti-apoptotic response in human cancer cells and p21 is believed to be the key molecule that mediates this anti-apoptotic response (Yu, Q. (2006) “Restoring p53-mediated apoptosis in cancer cells: New opportunities for cancer therapy” Drug Resist Updates, vol. 9, p. 19-25). It is also well known that p21 functions as a CDK inhibitor. Thus, manipulation of p21 level appears to be a feasible approach for modulating chemotherapeutic response (Weiss, R. H. (2003) “p21Waf1/Cip1 as a therapeutic target in breast and other cancers” Cancer Cell, vol. 4, p. 425-429).

In its effort to identify additional p53 targets that could be useful to influence the fate of the cell upon DNA damage, for example caused by a chemotherapeutic agent, the inventors observed here that RPS27L, which encodes the ribosomal protein S27-like (RPS27L) with a previously unknown function, is regulated by p53 (see Example 1 for further details). The results described in Example 1 show that RPS27L expression is up-regulated by p53 through direct DNA binding. It was further found in this invention that RPS27L protein expression depends on the type of stress for p53 activation. While its induction in ADR-treated cells is associated with the cell cycle arrest response, the decrease of RPS27L protein levels upon 5-FU treatment correlates with a strong apoptosis response (see Example 2 for further details). This means, for example, that in the absence of RPS27L, DNA damage, caused for example by a chemotherapeutic agent, induces apoptosis instead of cell cycle arrest. RPS27L therefore appears to function as a control switch to determine cellular outcome following p53 activation.

Thus, one aspect of the present invention provides a method of sensitizing a cell to cancer treatment comprising modulating the activity of a nucleic acid which encodes the RPS27L protein or the RPS27 protein itself by means of a respective compound that is able to modulate the gene activity of a RPS27L encoding nucleic acid or that is able to modulate the RPS27L protein. The cell to be subjected to the sensitizing can be any given cell and is typically a mammalian cell. The mammalian cell can, for example, be from a human, a mouse, a rat, a dog, a cat, a pig or cow, meaning that for example, a human patient or a mouse or any other mammalian can be treated using the sensitization method of the present invention.

In Example 3 the inventors have demonstrated that decreasing the level of RPS27L protein converts p53-dependent DNA damage response from growth arrest to cell death. Accordingly, the method of the present invention can, for example, be used to induce apoptosis in a malignant cell by modulating the activity of the nucleic acid which encodes the RPS27L protein.

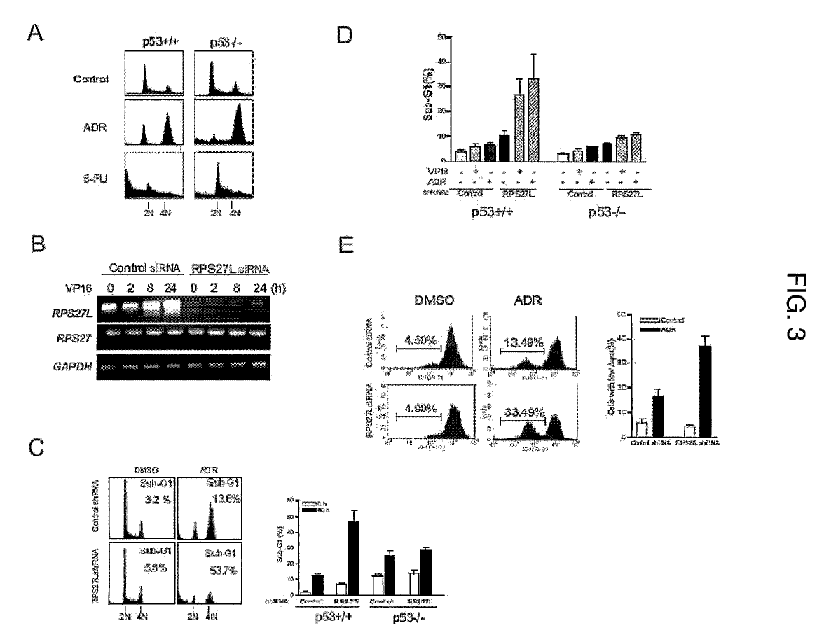

This approach is further illustrated in the following. In HCT116 cells, it is known that the DNA damaging agent ADR (a topoisomerase inhibitor used in chemotherapeutic cancer therapy) mainly induces p53-dependent cell cycle arrest (Bunz, F., Hwang, P. M., et al. (1999) “Disruption of p53 in human cancer cells alters the responses to therapeutic agents” J Clin Invest, vol. 104, p. 263-269; Tan, J., Zhuang, L., et al. (2005) “Pharmacologic modulation of glycogen synthase kinase-3beta promotes p53-dependent apoptosis through a direct Bax-mediated mitochondrial pathway in colorectal cancer cells” Cancer Res, vol. 65, p. 9012-9020). When decreasing the level of RPS27L protein in HCT116 cells and repeating the treatment with ADR, the cells undergo marked cell death instead of switching into the cell cycle arrest (see FIG. 3A and Example 3 for more details). The same effect can be observed when conducting the same experiment using etoposide phosphate (VP16®, another topoisomerase inhibitor) or other cell lines, like U2OS, Saos-2 and SH-SY5Y. Following this approach in the treatment of cancer, cells can be sensitized, i.e. they are rendered more susceptible, to a treatment with, for example, one or more chemotherapeutic anti-cancer drugs, when the level of RPS27L protein and also RPS27L mRNA coding for RPS27L protein is decreased. Thus, faced with the DNA damage due to the use of cytotoxic agents (like in anti-cancer chemotherapy) a malignant cell rather undergoes apoptosis than DNA repair when RPS27L protein level is depleted. Sensitization of a malignant cell by use of the method of the present invention avoids the use of high doses of chemotherapeutic anti-cancer drugs because the pathway to cell repair will be obstructed by decreasing the level of RPS27 protein level in the malignant cell. Accordingly, already low amounts of anti-cancer drugs provide effective treatment of tumors. The use of lower amounts of anti-cancer drugs evidently result in less severe or even no side effects which can normally be observed with the drugs usually used in anti-cancer chemotherapy.

A compound capable of modulating the activity of the nucleic acid which encodes the RPS27L protein may be a nucleic acid molecule that is able to influence the activity of the coding nucleic acid. Accordingly, in one embodiment the method of the present invention includes modulating the activity of the nucleic acid which encodes the RPS27L protein by administering a suitable nucleic acid such as an oligonucleotide. This oligonucleotide has the effect that upon administering it to a cell, preferably a malignant cell, of eukaryotic or even more precise a mammal cell, like a human malignant cell, will cause a modulation of the activity of the nucleic acid which encodes RPS27L protein. In one aspect the modulation of the activity of the nucleic acid encoding RPS27L protein means decreasing or increasing the level of this nucleic acid.

Nucleic acid molecules such as oligonucleotides that are able to modulate the activity of the nucleic acid encoding RPS27L protein can, for example, be an RNAi agent or an antisense nucleotide molecule. Introducing and transcribing an antisense nucleotide molecule into the cell leads to synthesis of a RNA molecule which is complementary to the mRNA molecule coding for RPS27L. This RNA molecule (i.e. antisense RNA) then provides a genetic tool that can be used for inhibiting the expression of the mRNA coding for RPS27L. The antisense molecule does of course not have to be able to provide a RNA molecule that is complementary to the entire RNA molecule coding for RPS27L but it is also sufficient to provide fragments of the antisense RNA to inhibit the expression of the mRNA coding for RPS27L. Antisense technology has become an established methodology (see for example, Weiss, B. (ed.): Antisense Oligodeoxynucleotides and Antisense RNA: Novel Pharmacological and Therapeutic Agents, CRC Press, Boca Raton, Fla., 1997, or Crooke, S. T. Progress in Antisense Technology, Annual Review of Medicine, February 2004, Vol. 55: Page 61-95), and the design of respective antisense nucleotide molecules is within the skill of the person of average knowledge in the art.

In addition or as an alternative to antisense nucleotide molecules, an RNAi agent (i.e., an interfering ribonucleic acid) can also be used as compound that modulates the activity of a nucleic acid encoding the RPS27L protein. The use of interfering ribonucleic acids such as interfering RNAs, short hairpin RNAs and micro RNAs has become a powerful tool to “knock down” specific genes. RNAi methodology makes use of gene silencing or gene suppression through RNA interference (RNAi), which occurs at the posttranscriptional level and involves mRNA degradation. RNA interference represents a cellular mechanism that protects the genome. siRNA and miRNA molecules mediate the degradation of their complementary RNA by association of the siRNA with a multiple enzyme complex to form what is called the RNA-induced silencing Complex (RISC). The siRNA or miRNA becomes part of RISC and is targeted to the complementary RNA species which is then cleaved. siRNAs are perfectly base paired to the corresponding complementary strand, while miRNA duplexes are imperfectly paired. Activation of RISC leads to the loss of expression of the respective gene (for a brief overview see Zamore, P. D. and Haley, B. (2005) “Ribo-gnome: The Big World of Small RNAs” Science, vol. 309, p. 1519-1524). Interfering ribonucleic acids may not exceed about 100 nt in length, and typically does not exceed about 75 nt length. Where the interfering ribonucleic acid is a duplex structure of two distinct ribonucleic acids hybridized to each other, e.g., a siRNA, the length of the duplex structure typically ranges from about 15 to 30 bp, usually from about 15 to 29 bp. Where the RNAi agent is a duplex structure of a single ribonucleic acid that is present in a hairpin formation, i.e., a shRNA, the length of the hybridized portion of the hairpin is typically the same as that provided above for the siRNA type of agent or longer by 4-8 nucleotides.

An illustrative example of a siRNA molecule that can be used in the method of sensitizing a cell to cancer treatment of the present invention comprises or has the 19 base pair long nucleotide sequence depicted in SEQ ID NO: 1 (see also Example 3). Thus, the agents used in the method of the present invention can be used in therapy for the treatment or prevention of cancer.

Where the mammalian malignant target cells are in vivo, the oligonucleotide that is used in the method of the present invention can be administered to the mammalian host using any convenient protocol which is known to a person skilled in the art. The following discussion provides a review of representative nucleic acid administration protocols that may be employed. The nucleic acids may be introduced into tissues or host cells by any number of routes, including viral infection, microinjection, or fusion of vesicles.

Jet injection may also be used for intra-muscular administration, as described by Furth, P. A., Shamay, A., et al. (1992) “Gene transfer into somatic tissues by jet injection” Anal Biochem, vol. 205, p. 365-368. The nucleic acid may be coated onto gold microparticles and delivered intradermally by a particle bombardment device or “gene gun” as described in the literature (see, for example, Tang, D. C., De Vit, M., et al., (1992) “Genetic immunization is a simple method for eliciting an immune response” Nature, vol. 356, p. 152-154), where gold microparticles are coated with the DNA, then bombarded into skin cells. The use of nanoparticles for delivering siRNA is another suitable approach for cell-specific targeting. This method has been described for example by Weissleder, R., Kelly, K., et al. (2005) “Cell-specific targeting of nanoparticles by multivalent attachment of small molecules” Nature Biotech, vol. 23, p. 1418-1423.

Another illustrative example of delivering an oligonucleotide used in the method of the present invention into selected cells in vivo is its non-covalent binding to a fusion protein of a heavy-chain antibody fragment (Fab) and the nucleic acid binding protein protamin (Song, E., Zhu, P., et al. (2005) “Antibody mediated in vivo delivery of small interfering RNAs via cell-surface receptors” Nature Biotech, vol. 23, p. 709-717). Another illustrative example of delivering a siRNA molecule into selected cells in vivo is its encapsulation into a liposome. Morrissey, D., Lockridge, J., et al. “Potent and Persistent In Vivo Anti-HBV Activity of Chemically Modified siRNAs” Nature Biotech (2005), vol. 23, p. 1002-1007) for instance used a stable nucleic acid-lipid-particle, coated with a polyethylene glycol-lipid conjugate, to form liposomes for intravenous administration. In the present invention the Lipofectamine 2000 system (Invitrogen) was exemplarily used to transfect cells with the nucleic acid sequence encoding the siRNA and shRNA (for further details see Example 3).

Yet a further example of delivering an RNAi agent to a selected malignant target cell is the use of a biological vehicle such as a bacterium or a virus (e.g. adenovirus) that includes the respective nucleic acid molecule. Xiang, S., Fruehauf, J., et al. (2006) “Short hairpin RNA-expressing bacteria elicit RNA interference in mammals” Nature Biotech, vol. 24, p. 697-702, have for instance used this approach by administering the bacterium E. coli, which transcribed from a plasmid inter alia both shRNA and invasin, thus permitting entry into mammalian cells and subsequent gene silencing therein.

Expression vectors may be used to introduce an oligonucleotide used in the method of the present invention into the desired cells. In addition, the oligonucleotide used in the method of the present invention can be fed directly to, injected into, the host organism containing the target gene, i.e. RPS27L. The oligonucleotide may be directly introduced into the cell (i.e., intracellularly); or introduced extracellularly into a cavity, interstitial space, into the circulation of an organism, introduced orally, etc. Methods for oral introduction include direct mixing of RNA with food of the organism. Physical methods of introducing nucleic acids include injection directly into the cell or extracellular injection into the organism of an RNA solution. The agent may be introduced in an amount which allows delivery of at least one copy per cell. Higher doses (e.g., at least 5, 10, 100, 500 or 1000 copies per cell) of the agent may yield more effective inhibition; lower doses may also be useful for specific applications.

As previously mentioned, the cancer treatment referred to in the method of the present invention can be chemotherapy in which a chemotherapeutic agent is used. The chemotherapeutic agent used in the method of the present invention include, but is not limited to, an alkylating agent, an antimetabolite, an antimitotic, a topoisomerase inhibitor, a platinum containing compound, hormones, signalling inhibitor, a monoclonal antibody, a biologic response modifier or an differentiating agent.

In general, chemotherapeutic agents act on different biochemical processes in certain phases of the cell cycle. For example, antimetabolites, like 5-fluorouracil and folic acid antagonists, primarily block the DNA-synthesis and thus act in the S-phase of the cell. Colchicin and vincristin inhibit mitosis in the M-phase of the cell. Other chemotherapeutic agents act in different phases of the cell cycle.

Examples for alkylating agents comprise cyclophosphamide, chlorambucil and melphalan. These compounds form chemical bonds with the DNA and cause breaks in DNA and errors in the replication of the DNA. Examples for antimetabolites comprise methotrexate, cytarabine, fludarabine, 6-mercaptopurine and 5-fluorouracil (5-FU). These compounds block the synthesis of DNA. Examples for antimitotics include vincristine, colchicin, paclitaxel and vinorelbine. These compounds block the division of cancer cells. Examples for topoisomerase inhibitors include, but are not limited to, doxorubicin (adriamycin, ADR), etoposide phosphate (VP16®) and irinotecan. These compounds prevent DNA synthesis and repair through blockage of enzymes called topoisomerases. Examples for platinum containing compounds/derivatives include cisplating and carboplatin. Such compounds form bonds with DNA causing breaks. Examples for compounds that are used for hormonal cancer therapy include, but are not limited to, tamoxifen and bicalutamide. For example, tamoxifen blocks the action of estrogen (in breast cancer) whereas bicalutamide blocks androgen action (in prostate cancer). An example of a signalling inhibitor is the compound imatinib. Imatinib blocks signal for cell division in chronic myeolytic leukemia. Illustrative examples of monoclonal antibodies include rituximab, trastuzumab and gemtuzumab ozogamicin. Rituximab causes cell death through binding to cell surface receptor on lymphocyte-derived tumors. Trastuzumab blocks the growth factor receptor on breast cancer cells and gemtuzumab ozogamicin contains a specific antibody that attaches to a receptor found on leukemic cells and then delivers a toxic dose of its chemotherapeutic component to the leukemic cells. An example for a biological response modifier is interferon-alpha which exact biochemical function in this regard is still unknown. An example for a differentiating agent is tretinoin which induces differentiation and death of leukemic cells.

In another embodiment the chemotherapeutic agent may be adriamycine (doxorubicine), nutlin-3, etoposide phosphate (VP16®) or 5-fluorouracile. It is of course also possible to use combinations of different chemotherapeutic agents together with a compound described here capable of modulating the activity of the nucleic acid which encodes the RPS27L protein.

It should be noted that all chemotherapeutic agents, directly or indirectly, cause DNA damage which again might affect the cell death by apoptosis. Due to the activation of the apoptotic pathway of the cell, modulating the activity of the nucleic acid encoding RPS27L can influence the cell fate. In case of a tumor, the desired approach would be to modulate the activity of RPS27L in a way that the malignant cell enters the apoptotic pathway rather then cell arrest or senescence. Thus, the combined effect of RNAi agent or antisense nucleotide molecule referred to in the method of the present invention depends not on a direct interaction of the chemotherapeutic agent with these oligonucleotides but rather on their influence on the same biochemical pathway activated upon cell damage, e.g. DNA damage. Those skilled in the art will thus appreciate that the present method of the invention preferentially induces apoptosis in malignant cells.

That RPS27L does indeed play a role in the p53-dependent biological pathway following DNA damaging is demonstrated by the results described in Example 4 to 6. The results described in this example further describe that RPS27L is a nuclear protein participating in DNA damage response and is recruited to a subset of DNA double-strand breaks.

In another aspect the present invention is directed to a pharmaceutical preparation including at least one chemotherapeutic agent as described above alone or together with for example, at least one oligonucleotide (for example, an RNAi agent and/or antisense nucleotide molecule) used in the method of the present invention.

The pharmaceutical preparation can be administered in a variety of formulations for therapeutic administration. More particularly, the pharmaceutical preparations of the present invention can be formulated into pharmaceutical compositions by combination with appropriate, pharmaceutical acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the pharmaceutical preparation can be achieved in various ways, including oral, buccal, rectal, parenteral, intraperitoneal, intradermal, transdermal, intracheal, etc., administration.

In pharmaceutical dosage forms, the pharmaceutical preparation of the present invention may be administered alone or in appropriate association, as well as in combination, with other pharmaceutical active compounds. The following methods and excipients are merely exemplary and are in no way limiting.

For oral preparations, the pharmaceutical preparations can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The pharmaceutical preparations can be formulated for injection by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The pharmaceutical preparations can be utilized in aerosol formulation to be administered via inhalation. The pharmaceutical preparations of the present invention can be formulated into pressurized acceptable propellants such as dichlorodifluoromethane, propane, nitrogen and the like.

One may also administer the pharmaceutical preparation in a local rather than systemic manner, for example, via injection of the compound directly into a solid tumour, such as in a depot or sustained release formulation. Furthermore, a respective compound or pharmaceutical composition may be used in a targeted drug delivery system, for example, in a liposome coated with a tumour-specific antibody. Such liposomes may for example be targeted to and taken up selectively by a tumour.

In addition to the formulations described previously, the pharmaceutical preparations may also be formulated as a depot preparation. Such long acting formulations may be administered by implantation (for example subcutaneously or intramuscularly) or by intramuscular injection. Thus, for example, the pharmaceutical preparation may be formulated with suitable polymeric or hydrophobic materials (for example as an emulsion in an acceptable oil) or ion exchange resins, or as sparingly soluble derivatives, for example, as a sparingly soluble salt.

### EXPERIMENTAL EXAMPLE 1

### Genomic Analysis Identifies RPS27L as a Direct Transcriptional Target of p53

p53 exerts its tumor suppressor function predominately through transcriptional regulation on downstream targets (Vogelstein, B., Lane, D. and Levine, A. J. (2000), supra; Vousden, K. H. and Lu, X. (2002), supra). In previous work to identify additional p53 downstream targets through genome-wide mapping of p53 binding loci (Wei, C. L., Wu, Q., et al., (2006), supra), it was observed that RPS27L, which encodes the ribosomal protein S27-like (RPS27L) with a previously unknown function, was potentially regulated by p53. RPS27L is located on chromosome 15 of the human genome with the position 61235856 to 61237660.

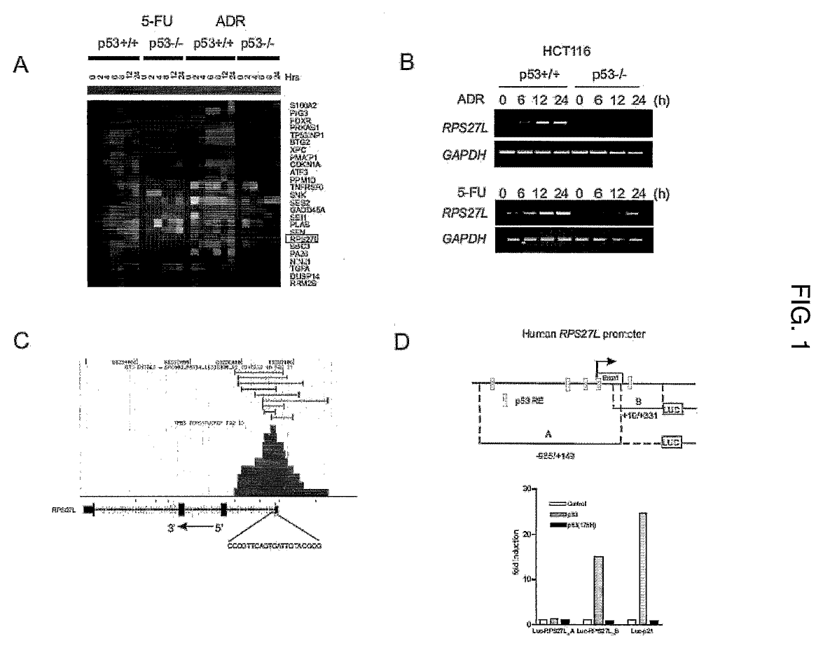

FIG. 1A depicts the microarray analysis showing a set of genes whose expression was induced by the DNA damaging agents adriamycin (ADR) and 5-fluorouracil (5-FU) in p53 wild-type HCT116 cells (human colorectal carcinoma cell line containing wild type p53) but not in the p53 null counterpart. In both treatments, RPS27L, together with bona fide p53 targets such as cdkn1a (encodes p21), Pmaid (encodes Noxa) and ccd3 (encodes Puma), was unregulated in a p53-dependent manner. To validate the microarray data, RT-PCR analysis was performed (FIG. 1B). FIG. 1B shows that RPS27L mRNA was induced following ADR or 5-FU treatment only in p53 wild-type HCT116 cells. These results demonstrate that there is a connection between the expression of RPS27L and the activation and expression of p53.

Furthermore, among the >500 p53 binding targets which the present inventors have previously identified in the human genome using chromatin immunoprecipitation (ChIP)-PET technology (Wei, C. L., Wu, Q., et al., (2006), supra), RPS27L was found to be strongly bound by p53 through a p53 binding motif located in the first intron (FIG. 1C). Thus, RPS27L expression appears to be up-regulated by p53 through direct DNA binding.

The physiological relevance of p53 binding in the RPS27L gene was also assessed here. In addition to the validated binding site located in the first intron, sequence analysis also identified four additional putative p53 responsive elements within 1.1 kb of the promoter region, as illustrated in FIG. 1D (upper panel). To assess whether any of these sites mediates the p53-dependent activation of RPS27L, the genomic DNA fragments spanning either the promoter region (Luc-RPS27L-A) or the p53 binding site in the first intron (Luc-RPS27L-B) were cloned into a luciferase reporter plasmid. When co-transfected into p53 null HCT116 cells together with a p53 expression plasmid, only the reporter containing the intronic sequences was activated by wild type p53 but not by a DNA binding-deficient p53 mutant (R175H) (FIG. 1D, lower pane). As a positive control, wild-type but not the mutant p53 also activates a reporter containing the p21 promoter. These results confirm that the p53 binding located in the first intron of RPS27L, as determined by ChIP analysis, is functional and confers the p53 responsiveness.

General procedures for carrying out nucleic acid modifications referred to in this and the following examples are described in Sambrook, et al., “Molecular Cloning: A Laboratory Manual”, Cold Spring Harbour Lab Publ. 1989 and Ausubel, et al. “Current Protocols in Molecular Biology” Grene Publishing Associates and Wiley-Interscience, 1987.

### EXAMPLE 2

### p53-Dependent RPS27L Protein Induction is Stimuli-Dependent

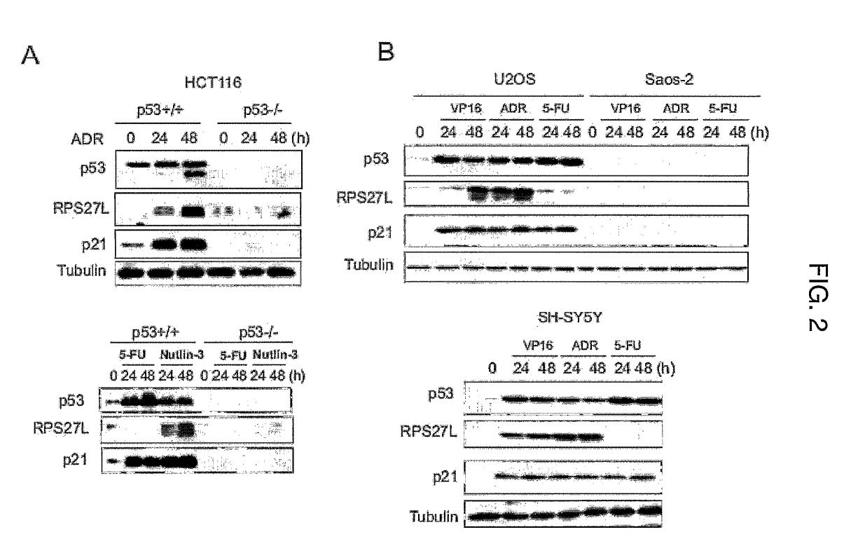

To investigate whether the p53-induced increase in RPS27L mRNA also gives rise to the increased protein levels, immunoblot analysis of p53 wild-type and p53 null HCT116 cells treated with ADR, 5-FU or nutlin-3, a small molecule MDM2 antagonist that directly activates p53 (Vassilev, L. T., Vu, B. T., et al. (2004) “In vivo activation of the p53 pathway by small-molecule antagonists of MDM2” Science, vol. 303, p. 844-848) was performed. ADR and nutlin-3 treatment resulted in the accumulation of p53 and p21. An antibody raised against RPS27L detected increased RPS27L protein levels over time following ADR or nutlin-3 treatment (FIG. 2A). However, 5-FU-induced upregulation of RPS27L mRNA did not give rise to increased protein level. Instead, RPS27L protein level was downregulated, which was in striking contrast to the increased p21 protein expression, along with p53 activation. To extend this observations to other cell lines and to other p53 stimuli, U2OS (human osteosarcoma cell line) and SH-SY5Y (human neuroblastoma cell line) cells (p53 wild type) as well as Saos-2 cells (human osteosarcoma cell line, p53-deficient) were treated with ADR, etoposide phosphate (VP16®) and 5-FU (FIG. 2B). Again, ADR and etoposide phosphate (VP16®) induced p53-dependent upregulation of RPS27L protein but 5-FU induced its downregulation. Collectively, these results indicate that p53-induced RPS27L protein expression depends on the type of stress leading to p53 activation. Obviously, 5-FU treatment activates a posttranslational mechanism that causes RPS27L protein downregulation in spite of the increased mRNA expression.

### EXAMPLE 3

### RPS27L Depletion Converts p53-Dependent DNA Damage Response from Growth Arrest to Cell Death

In HCT116 cells, it is known that the DNA damaging agent ADR induces p53-dependent cell cycle arrest (increased number of hyperploid cells (4N)), whereas 5-FU treatment triggers p53-dependent apoptosis (Bunz, F., Hwang, P. M., et al. (1999), supra; Tan, J., Zhuang, L., et al. (2005), supra) (FIG. 3A). Since increased RPS27L protein expression is correlated with the cell cycle arrest phenotype, the inventors next set out to determine whether RPS27L knockdown in HCT116 cells will render apoptosis rather than cell cycle arrest upon ADR treatment. To achieve this aim, the inventors silenced RPS27L expression using small interfering RNA (siRNA) of SEQ ID NO: 1. The targeted sequence for siRNA was efficient and specific as it nearly completely ablated RPS27L expression and prevented its induction after DNA damage, while having no effect on closely-related RPS27 (FIG. 3B). To facilitate the study, the inventors created a HCT116 cell line stably expressing either the short hairpin of SEQ ID NO: 1 to deplete RPS27L (RPS27L shRNA) or a non-specific control shRNA (control shRNA) in both p53 wild-type and null backgrounds and investigated their cellular responses to ADR. The control shRNA was obtained from Dharmacon Inc, Lafeyette, Colo., USA)

In p53 wild-type HCT116 cells expressing the control shRNA, ADR treatment for 48 h resulted primarily in a growth arrest response, while RPS27L-depleted cells receiving the same treatment underwent marked cell death (FIG. 3C). This effect appeared to be p53-dependent, as RPS27L depletion in p53 null HCT116 cells did not give rise to the same result. Knockdown of RPS27L through transient siRNA transfection also induced a marked increase in cell death upon ADR or etoposide phosphate (VP16®) treatment in HCT116 cells but not in p53 null counterparts (FIG. 3D). Similar effects were also seen in lung carcinoma A549 and osteosarcoma U2OS cells (data not shown), indicating that the increased cell death response to DNA damaging agents is a general feature of RPS27L deficiency.

To further define the nature of this cell death response the inventors used another assay, namely flow cytometric detection of cells stained with JC-1. The JC-1-staining identifies cell death events as a result of loss of mitochondria membrane potential (ΔΨm). As shown in FIG. 3E, ADR treatment of RPS27L shRNA cells resulted in a marked decrease in ΔΨm compared to the control cells (33.5% versus 13.5%), indicating an apoptotic cell death involving mitochondrial dysfunction. Hence, loss of RPS27L results in apoptosis rather than cell cycle arrest in response to p53 activation by DNA damage.

This experiment clearly demonstrates the usability of RPS27L as means for regulating the cell fate after DNA damaging. Modulating the activity of the nucleic acid encoding RPS27L by increasing or decreasing its transcription or translation allows to direct malignant cells into cell death when treating them with a chemotherapeutic agent as demonstrated in this example. The use of regulatory molecules (i.e. oligonucleotides that are used in the method of the present invention, like RNAi agents or antisense nucleotide molecules) allows influencing the transcription or translation (i.e. expression) of the nucleic acid encoding RPS27L.

### EXAMPLE 4

### RPS27L is a Nuclear Protein that Forms Nuclear Foci Upon DNA Damage

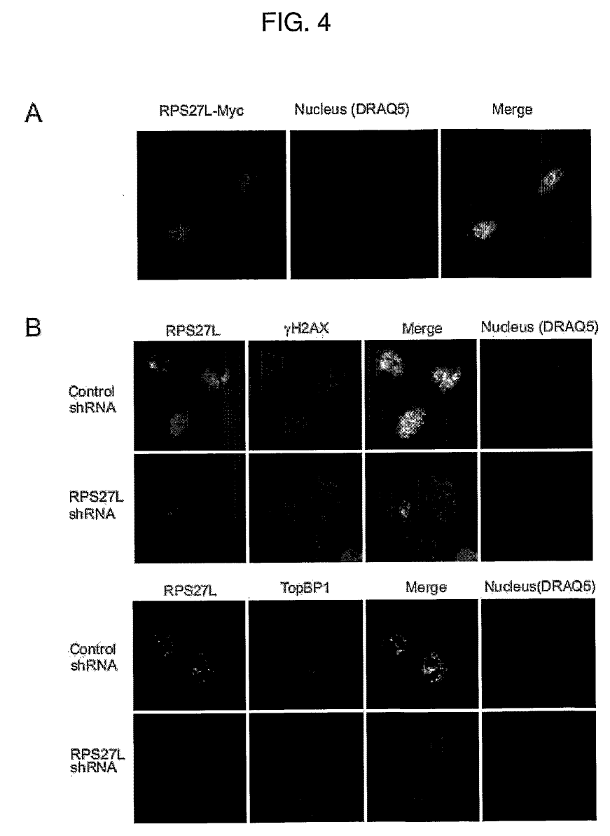

To further characterize the function of RPS27L in DNA damage response, the cellular localization of RPS27L was evaluated. c-Myc tagged RPS27L was over-expressed in HCT116 cells and expression was detected by immunofluorescence staining with an anti-c-Myc antibody. Ectopically expressed RPS27L localizes to the nucleus (FIG. 4A). To determine the cellular location of the endogenous RPS27L and to assess its response to DNA damage, the inventors next performed immunofluoresence studies using an anti-RPS27L antibody. Both wild-type and RPS27L-depleted HCT116 cells were treated with etoposide phosphate (VP16®) for 16 hours prior to fixation. Because of the low level of RPS27L in untreated cells, the inventors obtained low nuclear staining in these cells (data not shown). Upon etoposide phosphate (VP168) treatment, the inventors detected a nuclear foci-like staining pattern in HCT116 cells with anti-RPS27L (FIG. 4B). In addition, RPS27L partially colocalized with phosphorylated histone H2AX (γ-HA2X). γ-H2AX is a histone phosphorylated at sites of DNA double strand breaks (DSB) and is the hallmark of DSB (Rogakou, E. P., Pilch, D. R., et al., (1998) “DNA double-stranded breaks induce histone H2AX phosphorylation on serine 139” J Biol Chem, vol. 273, p. 5858-5868). Depletion of RPS27L expression abolished this co-localization, although it had no effect on γ-HA2X foci formation. These data suggest that RPS27L is a nuclear protein participating in DNA damage response and is recruited to a subset of DNA double-strand breaks.

### EXAMPLE 5

### RPS27L Depletion Results in Functional Defects in the DNA Damage Checkpoint and DNA Repair

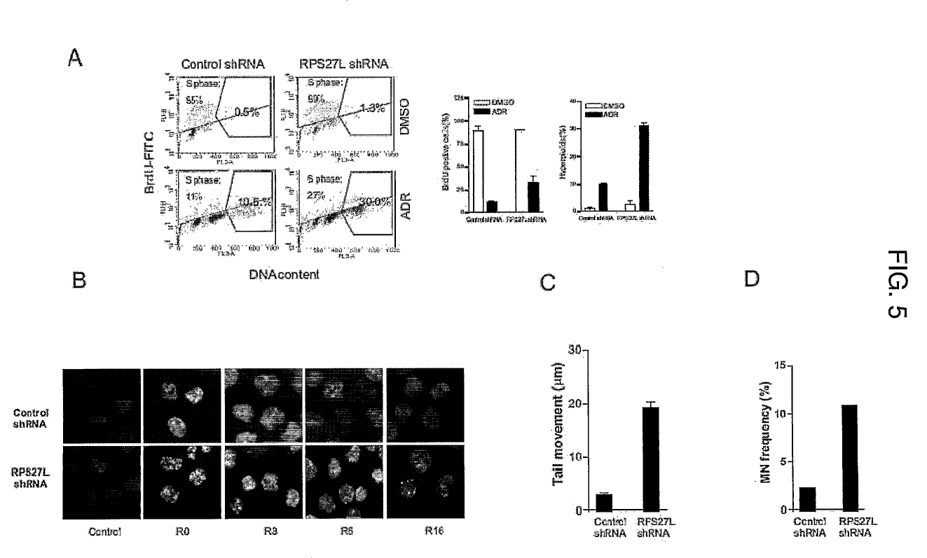

Given the increased sensitivity to DNA damage upon RPS27L depletion, the inventors next investigated whether loss of RPS27L could impair the DNA damage checkpoint. FACS analysis with 7-amino-actinomycin D staining (for total DNA content) and bromodeoxyuridine (BrdU) labeling (for DNA synthesis) failed to detect a difference in DNA synthesis in control versus RPS27L-depleted HCT116 cells before DNA damage. However, parental cells underwent a dramatic decrease in DNA synthesis (from 85% to 11%) 24 hours after ADR treatment, whereas in HCT116 cells lacking RPS27L, this decrease in DNA synthesis was partially rescued (from 86% to 27%) (FIG. 5A). In addition, RPS27L-depleted HCT116 cells underwent a substantial accumulation of cells with a hyperploid DNA content (>4.v) compared with the control cells (30% vs 10.5%). These findings suggest that loss of RPS27L might confer a DNA damage cell cycle checkpoint defect leading to chromosome instability.

A deficient DNA damage checkpoint is expected to increase DNA damage. γ-H2AX foci are considered to be sensitive indicators of double strand breaks (DSB). We looked for γ-H2AX focus formation after etoposide phosphate (VP16®) treatment and monitored the reversibility post-VP16 washout. FIG. 5B shows that a one hour treatment with VP16 induced prominent DSB foci, as evidenced by strong γ-H2AX staining. In control cells, γ-H2AX staining was decreased over time, and by 16 hours after VP16 removal the —H2AX staining was almost undetectable, suggesting a proficient DNA repair process in these cells. In contrast, RPS27L-depleted HCT116 cells displayed a sustained γ-H2AX staining, pointing to the increased DNA damage in these cells. These results support our hypothesis of a protective role for RPS27L in DNA damage response.

In order to examine DNA damage directly, the comet assay was used next. The comet assay is a single cell gel electrophoresis assay in which damaged DNA migrates to form a “comet tail” that is proportional to the amount of DNA damage (Collins, A. R. (2004) “The comet assay for DNA damage and repair: principles, applications, and limitations” Mol Biotechnol, vol. 26, p. 249-261). Cells were incubated with ADR for 24 h and were harvested and processed for the comet assay. As shown in FIG. 5C, ADR-induced DNA damage was significantly enhanced in RPS27L-depleted cells, which is consistent with a role for RPS27L in DNA repair. We then investigated whether elevated DNA damage has caused chromosome instability. To test this possibility, micronuclei (MN) analysis was used, as it proves to be a reliable indicator of chromosomal damage and genomic instability (Fenech, M. (2005) “In vitro micronucleus technique to predict chemosensitivity” Methods Mol Med, vol. 111, p. 3-32; Poonepalli, A., Balakrishnan, L., et al. (2005) “Lack of poly(ADP-ribose) polymerase-1 gene product enhances cellular sensitivity to arsenite” Cancer Res, vol. 65, p. 10977-10983). There was increased micronucleus frequency in RPS27L-depleted cells upon ADR treatment, further supporting the effect of RPS27L loss on chromosome stability (FIG. 5D). Overall, these experiments demonstrate that loss of RPS27L leads to increased DNA damage and chromosome breaks after treatment of cells with ADR.

### EXAMPLE 6

### RPS27L Depletion Impairs p21 Accumulation Upon DNA Damage, which Confers Hypersensitivity to DNA Damage

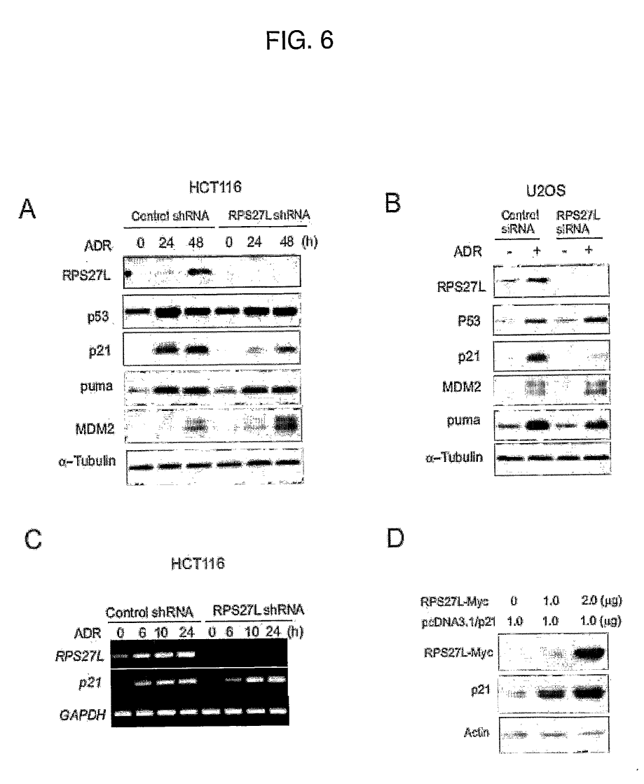

As RPS27L depletion sensitizes only p53 wild-type cells to DNA damage, the inventors next assessed the effect of RPS27L loss on p53 signaling pathway. The effects of RPS27L depletion on p53 and its downstream targets p21, puma and MDM2 was examined by immunoblot analysis. It was found that depletion of RPS27L had no significant effect on ADR-induced p53 activation (FIG. 6A). However, p21 accumulation in response to ADR was significant reduced in RPS27L-depleted cells compared to the control cells. By contrast, p53-dependent activation of Puma and MDM2 were not decreased upon RPS27L loss. This result suggests a selective impairment of p21 induction by p53 upon loss of RPS27L. Similar to the situation in HCT116 cells stably depleted of RPS27L, the inventors found a substantial decrease in p21 protein level in U2OS cells in which RPS27L was knocked down by transient transfection (FIG. 6B). These results indicate that the impaired p21 protein accumulation upon loss of RPS27L was not cell type specific.

It was further found that the reduced p21 protein level was not due to inhibited p21 transcription since p21 mRNA levels were not significantly changed in RPS27L-depleted cells compared to the control cells (FIG. 6C). To obtain direct evidence that RPS27L regulates p21 protein expression, the inventors co-transfected a p21-expressing vector (pcDNA) with increasing amounts of an RPS27L-expressing vector into HCT116 cells and the result showed that RPS27L overexpression markedly increased p21 protein expression in a dose-dependent manner (FIG. 6D). Collectively, these findings suggest that RPS27L positively regulates p21 protein expression. Loss of RPS27L results in attenuated p21 protein induction following DNA damage.

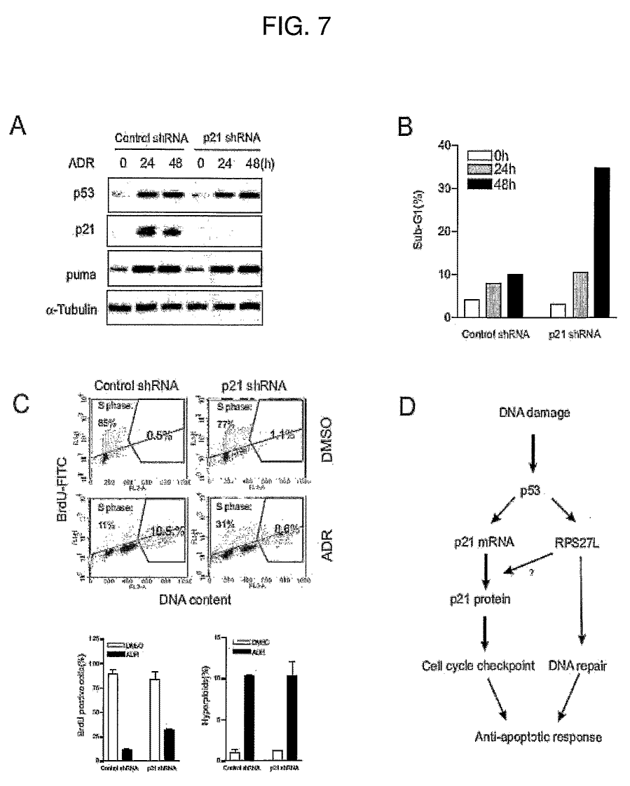

It is well-known that p21-deficient cells are defective in cell cycle arrest and are more sensitive to DNA damaging agents (Bunz, F., Kobayashi, R., et al. (1993) “cDNAs encoding the large subunit of human replication factor C” Proc Natl Acad Sci USA, vol. 90, p. 11014-11018; Fan, S., Chang, J. K., et al. (1997) “Cells lacking CIP1/WAF1 genes exhibit preferential sensitivity to cisplatin and nitrogen mustard” Oncogene, vol. 14, p. 2127-2136). To establish a functional role for p21 decrease in the phenotypic changes upon RPS27L depletion, the inventors created an HCT116 cell line stably expressing p21 shRNA (a commercial product obtained from Dharmacon Inc, Lafayette, Colo., USA was used as p21 shRNA). In these cells, p21 expression and its induction after ADR treatment were nearly completely abrogated (FIG. 7A). In response to ADR treatment, p21 shRNA cells underwent massive cell death, while the control cells remained growth arrested (FIG. 7B). In addition, BrdU staining indicated that the inhibition of BrdU incorporation after ADR treatment was reduced in p21-depleted cells (FIG. 7C). Thus, p21-depleted cells resembled the apoptotic and cell cycle phenotype of RPS27L-depleted cells. Further knockdown of RPS27L in p21-depleted cells did not render additional effects on the level of cell death or Brdu staining in response to ADR (data not shown). These results suggest that the impaired p21 protein accumulation in response to DNA damage in RPS27L-deficient cells is sufficient to confer the defect in cell cycle arrest and hypersensitivity to DNA damage.

However, no increased hyperploid population in p21-depleted cells after ADR treatment (FIG. 7C) was observed. Furthermore, comet assays revealed that increased DNA damage in p21-depleted cells was not as evident as in RPS27L-depleted cells (data not shown). Thus, the defective DNA repair and increased chromosome instability upon RPS27L loss appeared to be independent of p21 attenuation. The inventors propose that the induction of RPS27L by p53 results not only in growth arrest in response to DNA damage through enhanced p21 protein accumulation, but also promotes DNA repair and chromosome stability through additional mechanisms (FIG. 7D). Collectively, these functions effectively protect against the DNA damage response.

### Details to the Experimental Procedures Used in the Above Referenced Examples

### Cell Culture and Drugs

The human colon carcinoma cell line HCT116 and its p53 knock-out derivate cells were kindly provided by Dr. Bert Vogelstein. HCT116 cells can also be purchased under the ATCC number: CCL 247. The human osteosarcoma cell lines U2OS and Saos-2 can be purchased under the following ATCC numbers: U2OS, ATCC HTB-96; Saos-2, ATCC HTB-85 and SH-SY5Y, ATCC CRL-2266. Cells were grown in DMEM supplemented with 10% fetal bovine serum and penicillin-streptomycin (Invitrogen). Adriamycin, etoposide phosphate (VP16®) and 5-fluorouracil were purchased from Sigma-Aldrich.

### Flow Cytometry

Cells cycle analysis was performed by DNA content quantification. The cells were fixed with 70% ethanol and stained with propidium iodide (50 μg/ml) staining. The stained cells were analyzed by FACScalibur (BD Bioscience). For the BrdU incorporation assay and mitochondrial membrane potential detection, the BrdU Flow Kit and JC-1 staining kit (both from BD Bioscience) were used, respectively, following the instruction manual. Stained cells were analyzed by FACScalibur (BD Bioscience) and quantified by using CellQuest software (BD Bioscience).

### Gene Silence by RNA Interference

The siRNA oligo targeting RPS27L (SEQ ID NO: 1: ggttgctacaagattacta was purchased from Proligo, and transfection was conducted using Lipofectamine 2000 (Invitrogen) according to the manufacturer's information. To generate stable knock-down cell lines, the siRNA sequences were cloned into the pSIREN-RetroQ retroviral expression vector (BD Bioscience) according to the manufacturer's instruction. Virally infected cells were selected in a medium containing 2 μg/ml puromycin and individual drug-resistant clones were collected, pooled and expanded.

### Protein Analysis and Generation of Anti-RPS27L Antibody

Cells were harvested and lysed with RIPA buffer (50 mM Tris-HCl, pH7.4, 1 mM EDTA, 150 mM NaCl, 1% Nonidet P-40, 0.5% sodium deoxycholate and proteinase inhibitors). The lysates were clarified by centrifugation at 16,000×g for 15 minutes at 4° C. Protein concentrations were determined with the Bradford Protein Assay Kit (Bio-Rad). 20-50 μg protein samples were separated by SDS-PAGE, transferred onto an Immobilon membrane (Millipore) and blotted with antibodies. Anti-p53 and anti-p21 antibodies were from Santa Cruz; anti-MDM2 and anti-Puma antibodies were from Merck. The rabbit polyconal antibody to RPS27L was raised against a 14 amino acid peptide from human RPS27L (SEQ ID NO: 2: LHPSLEEEKKKHKK).

### Luciferase Report Assay

The promoter elements of RPS27L were cloned into the pGL3-luciferase vector (Promega). HCT116 p53−/− cells were plated in 24-well cell culture plates and co-transfected with the p53 expression vectors and RPS27L promoter plasmids. Twenty-four hours after transfection, the luciferase activities were measured using the Dual Luciferase system (Promega) as described (Kho, P. S., Wang, Z., et al. (2004) “p53-regulated transcriptional program associated with genotoxic stress-induced apoptosis” J Biol Chem, vol. 279, p. 21183-21192).

### Immunofluorescence Staining and Confocal Microscopy

The cells were seeded in 4-well or 8-well culture slides. After treatment, cells were fixed with 3.7% paraformaldehyde in PBS and permeabilized with 0.2% Triton-X100. Cells were sequentially incubated with primary antibodies (see above under “Protein Analysis and Generation of Anti-RPS27L Antibody”) and Alexa Fluor 488 or Alexa Fluor 546-conjugated secondary antibodies (Invitrogen) for 1 hour each and mounted in Fluorsave (Merck) mounting medium. DRAQ5 (Biostats, UK) was diluted in mounting medium for nuclear staining. The stained cells were examined by Zeiss LSM510 confocal microscopy.

### Plasmids

pcDNA4/RPS27L-Myc was generated by RT-PCR using normal colon tissue total RNA (Ambion), PowerScript Reverse Transcriptase (Clontech) and Platium PCR SuperMix High Fidelity (Invitrogen) with the primers GGTACCATGCCTTTGGCTAGAGATTT (Forward, SEQ ID NO: 3) and GAATTCTTAGTGTTGCTTTCTTCTAAATGA (Reverse, SEQ ID NO: 4). The PCR product and empty vector were digested with KpnI and EcoRI (NEB) and ligated with T4 ligase (NEB), followed by transformation and selection.

### Cytokinesis Blocked Micronucleus (CBMN) Assay

After treatment with ADR, cells were incubated with cytochalasin B (Sigma, 5 μg/ml) for an additional 22 h. The cells were then trypsinized and subsequently fixed using a combination of both Carnoy's fixative (acetic acid: methanol, 1:3) and 3-4 drops of formaldehyde (to fix the cytoplasm). Fixed cells were dropped onto clean slides and stained with 3 μg/ml Acridine Orange (which differentially stains cytoplasm and nucleus) (Hande, M. P., et al. (1996) “Induction and persistence of cytogenetic damage in mouse splenocytes following whole-body X-irradiation analysed by fluorescence in situ hybridization. II. Micronuclei.” Int J Radiat Biol.; vol. 70(4), p. 375-83; Hande, M. P., et al. (1997) “Induction and persistence of cytogenetic damage in mouse splenocytes following whole-body X-irradiation analysed by fluorescence in situ hybridization. III. Chromosome malsegregation/aneuploidy” Mutagenesis, vol. 12(3), p. 125-31). One thousand binucleated cells were scored for each sample.

### Alkaline Single Cell Gel Electrophoresis (Comet) Assay

Cells were treated with ADR with the doses mentioned above. The treated cells were subjected to a single cell gel electrophoresis (comet) assay as described earlier (Poonepalli, A., Balakrishnan, L., et al. (2005), supra) and stained with SYBR green dye. The tail moment of the comets was generated using the Metasystems (Germany) analysis software ‘Comet imager version 1.2’. Fifty randomly chosen comets were analyzed per sample. The extent of DNA damage observed was expressed as tail moment, which corresponded to the fraction of the DNA in the tail of the comet.

### RT-PCR Analysis

All RT-PCR was performed using the Titanium One Step RT-PCR Kit (BD Clontech). Primer sequences were:

p21, forward SEQ ID NO: 5:
5′-ATGTCAGAACCGGCTGGGGA-3′;
p21, reverse SEQ ID NO: 6:
5′-ATCACAGTCGCGGCTCAGCT-3′;
Puma, forward SEQ ID NO: 7:
5′-CGGACGACCTCAACGCACAGTA-3′;
Puma reverse SEQ ID NO: 8:
5-′AATTGGGCTCCATCTCGGGG-3′;
RPS27L, forward SEQ ID NO: 9:
5′-GTGACGACCTACGCACACGA-3′;
RPS27L, reverse SEQ ID NO: 10:
5′-GTGCTGCTTCCTCCTGAAGG-3′;
GAPDH, forward SEQ ID NO: 11:
5′-CAAAGTTGTCATGGATGACC-3′;
GAPDH, reverse SEQ ID NO: 12:
5′-CCATGGAGAAGGCTGGGG-3′.

The listing or discussion of a previously published document in this specification should not necessarily be taken as an acknowledgement that the document is part of the state of the art or is common general knowledge.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

1. A method of sensitizing a cell to cancer treatment comprising administering to said cell a compound capable of modulating the activity of a nucleic acid which encodes the RPS27L protein.

2. The method according to claim 1, wherein said cell is a eukaryotic cell.

3. The method according to claim 2, wherein said cell is a mammalian cell.

4. The method according to claim 3, wherein the mammalian cell is from a human, a mouse, a rat, a dog, a cat, a pig or cow.

5. The method according to claim 1, wherein modulating the activity of the nucleic acid which encodes the RPS27L protein comprises administering at least one oligonucleotide.

6. The method according to claim 5, wherein said oligonucleotide is an RNAi agent or an antisense nucleotide molecule.

7. The method according to claim 6, wherein said RNAi agent is an interfering ribonucleic acid.

8. The method according to claim 7, wherein said interfering ribonucleic acid is a siRNA or shRNA.

9. The method according to claim 8, wherein said shRNA comprises the nucleotide sequence depicted in SEQ ID NO: 1.

10. The method according to claim 1, wherein at least one chemotherapeutic agent is used in said cancer treatment.

11. The method according to claim 10, wherein said cancer treatment is a chemotherapy.

12. The method according to claim 10, wherein said chemotherapeutic agent is selected from the group consisting of alkylating agents, antimetabolites, antimitotics, topoisomerase inhibitors, platinum derivatives, hormonal therapies, signalling inhibitors, monoclonal antibodies, biologic response modifiers and differentiating agents.

13. The method according to claim 12, wherein the chemotherapeutic agent is selected from the group consisting of adriamycine (doxorubicine), nutlin-3, etoposide phosphate and 5-fluorouracile.

14. A method of sensitizing a cell against cancer treatment comprising administering to said cell a compound capable of inhibiting the activity of the RPS27L protein.

15. The method according to claim 15, wherein said cell is a eukaryotic cell.

16. The method according to claim 14, wherein said cell is a mammalian cell.

17. The method according to claim 14, wherein at least one chemotherapeutic agent is used in said cancer treatment.

18. The method according to claim 17, wherein said cancer treatment is a chemotherapy.

19. The method according to claim 17, wherein said chemotherapeutic agent is selected from the group consisting of alkylating agents, antimetabolites, antimitotics, topoisomerase inhibitors, platinum derivatives, hormonal therapies, signalling inhibitors, monoclonal antibodies, biologic response modifiers and differentiating agents.

20. The method according to claim 19, wherein said chemotherapeutic agent is selected from the group consisting of adriamycine (doxorubicine), nutlin-3, etoposide phosphate and 5-fluorouracile.

21. An expression vector comprising at least one oligonucleotide as defined in claim 5.

22. A pharmaceutical composition, comprising at least one RNAi agent or at least one antisense nucleotide molecule according to claim 6.

23. The pharmaceutical composition according to claim 22, comprising at least one RNAi agent and at least one antisense nucleotide molecule.

24. The pharmaceutical composition according to claim 22, further comprising at least one chemotherapeutic agent.

25. The pharmaceutical composition according to claim 22, further comprising a pharmaceutically acceptable delivery vehicle.

26. A method of treatment of cancer comprising administering to a patient a compound capable of modulating the activity of a nucleic acid which encodes the RPS27L protein for the preparation of a medicament for sensitizing a cell to cancer treatment.

27. A method of treatment of cancer comprising administering to a patient a compound capable of modulating the activity of a nucleic acid which encodes the RPS27L protein for the preparation of a medicament for sensitizing a cell to cancer treatment.


SEQUENCE LISTING (12 sequences)

SEQ ID NO: 1 - DNA, 19 bp, Artificial Sequence
  siRNA oligonucleotide targeting RPS27L
ggttgctacaagattacta

SEQ ID NO: 2 - protein, 14 aa, Homo sapiens
LHPSLEEEKKKHKK

SEQ ID NO: 3 - DNA, 26 bp, Artificial Sequence
  Forward primer
ggtaccatgcctttggctagagattt

SEQ ID NO: 4 - DNA, 30 bp, Artificial Sequence
  Reverse primer
gaattcttagtgttgctttcttctaaatga

SEQ ID NO: 5 - DNA, 20 bp, Artificial Sequence
  Forward primer against p21
atgtcagaaccggctgggga

SEQ ID NO: 6 - DNA, 20 bp, Artificial Sequence
  Reverse primer against p21
atcacagtcgcggctcagct

SEQ ID NO: 7 - DNA, 22 bp, Artificial Sequence
  Forward primer against Puma
cggacgacctcaacgcacagta

SEQ ID NO: 8 - DNA, 20 bp, Artificial Sequence
  Reverse primer against Puma
aattgggctccatctcgggg

SEQ ID NO: 9 - DNA, 20 bp, Artificial Sequence
  Forward primer against RPS27L
gtgacgacctacgcacacga

SEQ ID NO: 10 - DNA, 20 bp, Artificial Sequence
  Reverse primer against RPS27L
gtgctgcttcctcctgaagg

SEQ ID NO: 11 - DNA, 20 bp, Artificial Sequence
  Forward primer against GAPDH
caaagttgtcatggatgacc

SEQ ID NO: 12 - DNA, 18 bp, Artificial Sequence
  Reverse primer against GAPDH
ccatggagaaggctgggg